(19) United States
(12) Patent Application Publication
(10) Pub. No.: US 2004/0093062 A1
(43) Pub. Date: May 13, 2004

(54) Assembly with therapeutic action

(76) Inventor: Hendrik Glastra, Enschede (NL)

Correspondence Address: OSTROLENK FABER GERB & SOFFEN, 1180 AVENUE OF THE AMERICAS, NEW YORK, NY, 100368403

(21) Appl. No.: 10/472,643

(22) PCT Filed: Mar. 19, 2002
(86) PCT No.: PCT/NL02/00179

(30) Foreign Application Priority Data
Mar. 22, 2001 (NL) 1017672

(51) Int. Cl. A61F 2/06

(52) U.S. Cl. 623/1.11

(57) ABSTRACT

To obviate the problems which occur after a treatment of a stenosis by means of the usual dotter treatment followed by the placing of a stent which delivers a medically active substance, the invention proposes an implant assembly, to be fixed in a body vessel, with a section to be directed in the direction of the vessel axis and provided with means to act from therefrom on the surrounding tissue, in which this section, which can be fixed with at least one end by means of an anchoring element to the vessel wall, has a non-rigid structure, in such a way that it can adapt itself to the vessel wall configuration without any appreciable radial pressure on the surrounding tissue. Thus the surrounding tissue is not subjected to undesired pressures and resulting into a rapid recovery, without the formation of new tissue.

The invention relates to an implant assembly to be fixed in place in a body vessel, comprising a section which is directed in the direction of the vessel axis and is provided with means to act upon the surrounding tissue.

An implant assembly, to be fixed in place in a body vessel or body duct, which can deliver a therapeutically active substance to its direct environment is described in extenso in WO 91/12779 (Medtronics Inc.). This publication, the contents of which are still valid, describes in extenso the various aspects of the so-called “dotter”-treatment to remove a stenosis and the use of a therapeutically active stent which delivers in a controlled way a medicine (aspirin, heparin) to delay or even prevent restenosis which might occur after this treatment. The stent as described in this publication is, in the same way as stents of a later date and having the same structure and action, designed such that the anchoring thereof in the blood vessel is ensured by radially and outwardly expanding of the stent, thus exerting a omnidirectional radial pressure on the surrounding vessel wall.

It is known that the therapeutic action, to be obtained by such an implant, is in fact in practice in many cases very disappointing and that at the position of, and particularly under the implanted stent in many cases new tissue and coagulations are formed, which present, in fact, a serious danger for the health of the patient.

Placing a therapeutic active stent is not only done after a dotter treatment but also when one desires a local treatment of a vessel wall. The known stents which are used to this end are mostly self-expanding, having only a limited capacity for storing a therapeutic active substance, so that such a stent looses its effectiveness after a short time (for instance two weeks). The formation of new tissue is thus only halted for a short time period.

The invention is based on the insight that for obtaining an effective action of an implant assembly as discussed hereinabove the therapeutically active part thereof must bear against the inner wall of the body vessel—particularly a blood vessel—but that this part should bear against this wall while exerting forces on the inner surface of said vessel which are as weak as possible. To obtain this the invention proposes that this section can be anchored with at least one end by means of an anchoring element to the vessel wall and has a non-rigid structure, in such a way that it can adapt itself to the vessel wall configuration without exerting apprecible radial pressures on the surrounding tissue.

The anchoring element can be narrow as compared with the length of the section, so that its action upon the vessel wall, and therewith the detrimental by effect, is very limited. The section can lie freely and without exerting any pressure against the vessel wall. In fact the surgion has a very great freedom in choosing the material of the section—from a very “supple” to even flexible and elastically yielding material.

The section can have a cylindrical shape as known in itself, but is also possible that it is strip-shaped, being anchored with a number of similar sections along the circumference of the anchoring element. This anchoring element can be a radially expandable ring.

Embodiments as described above are suitable when the vessel is a blood vessel in which there is always a certain mean pressure and in which the current is alway unidirectional.

The embodiment as described in claim 11 has the advantage that, using a standard stent assembly initially without active material, the surgeon can decide in each case about the amount of therapeutically active material which the wants to be administered just before the treatment is started.

A preferred embodiment comprises two distant anchoring elements with the section or the sections thereinbetween. By a correct choice of the distance between these anchoring elements a close contact between the sections and the vessel wall, exerting a preetermined, preferably minimal radial pressure, is ensured. A major advantage lies in the fact that the anchoring elements can be positioned in healthy vessel parts and that even curved vessel parts can be bridged.

When so desired cut-outs can b provided in the section surface by means of which the blood can reach the vessel wall thereunder.

In a preferred embodiment the section consists of biologically degradable material; after the degradation thereof only the narrow anchoring elements remain in the vessel.

Positioning of the assembly does not pose any problem: on can use the usual positioning catheter, the anchoring element being an expandable metal ring, possibly made from memory metal, or a plastics ring which after expanding—by using an expanding balloon, and irradiating with, for instance, UV-radiation cures and thus retains its shape. By choosing the mutual distance of the anchoring elements the pre-tension in the section, and therewith the radial pressure exerted thereby on its surroundings, can be adjusted in a simple way to a desired value.

Of course the use of the assembly according to the invention is not limited to its positioning after a dotter treatment; the assembly can be used very effectively to treat local ailments for which a dotter treatment is unsuitable or superfluous. After the treatment with the assembly according to the invention a final stent can be placed at the treated part, as the inventive assembly uses only a minimal space, particularly when the section is made up from biological degradable material.

Known stents have the drawback that the radial pressure, necessary to position same, can result into the breaking up of the “plaque” so that parts thereof can migrate into the blood stream, sometimes with fatal results. With the narrow anchoring elements as proposed by the invention this danger is much smaller or even completely absent.

It is observed that EP 0 689 802 describes a blood vessel prothesis with a tubular body with at each end a radially expandable stent-shaped element that is meant to ensure the anchoring thereof in the blood vessel. EPO 680 733 describes a blood vessel prothesis with also at both ends a radially pretensioned anchoring ring. Both of these protheses are typically destined to bridge an aneurism; the inventive insight, as described herein above, cannot be derived therefrom particularly the prothesis bodies are not structured to deliver a therapeutically active substance for ionising radiation.

The invention is elucidated on the hand of the drawing. Therein show:

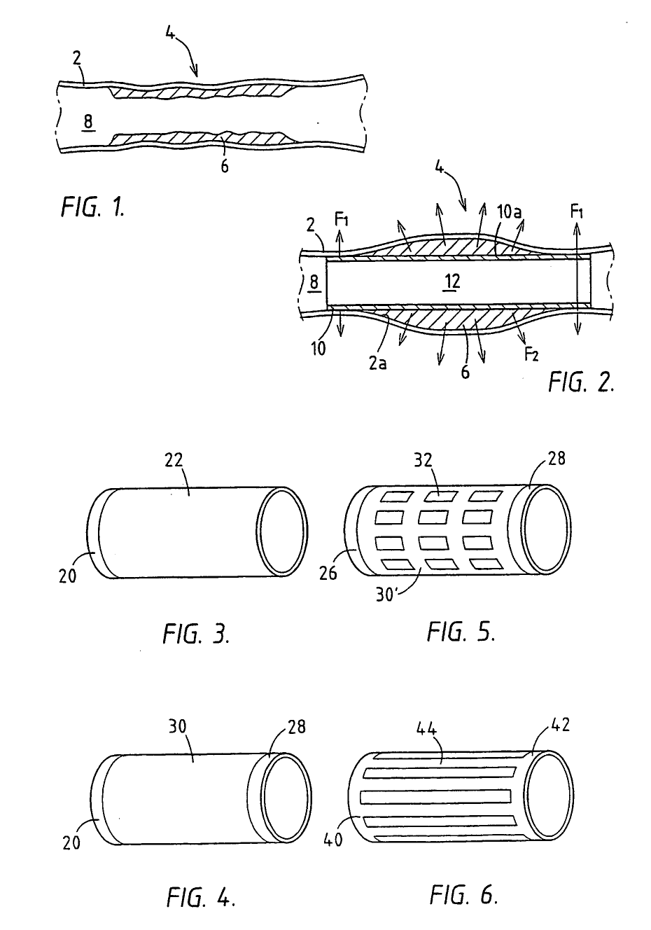

FIG. 1 longitudinal cross-section of a blood vessel with tissue remains therein, such as these typically remain after a dotter treatment;

FIG. 2 the radial pressures occurring in such a blood vessel after the placing therein of the prior art stent;

FIG. 3 a perspective view of a first embodiment of the assembly according to the invention;

FIG. 4 a perspective view of a preferred second embodiment of the invention;

FIG. 5 a perspective view of an embodiment according to FIG. 4 with cut-outs in the surface of the section;

FIG. 6 a perspective view of a third embodiment according to the invention;

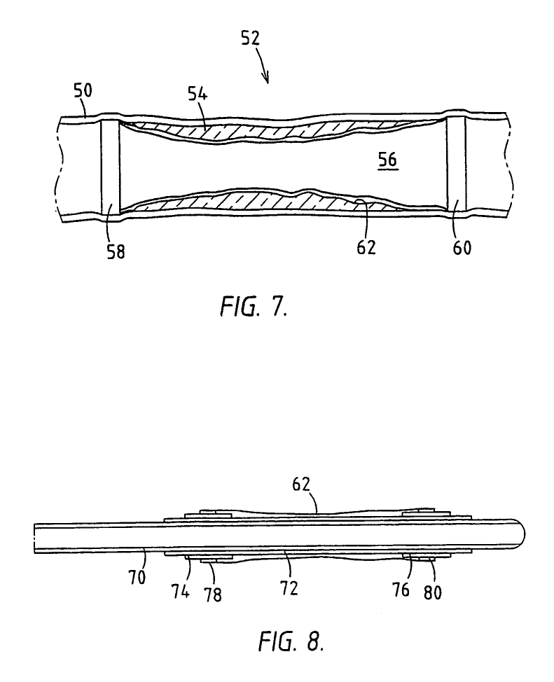

FIG. 7 a longitudinal cross-section through a part of a blood vessel with therein tissue remains after the positining of an assembly according to the invention;

FIG. 8 a schemacially longitudinal cross-section of the end of a positioning catheter suitable to position the assembly according to the invention;

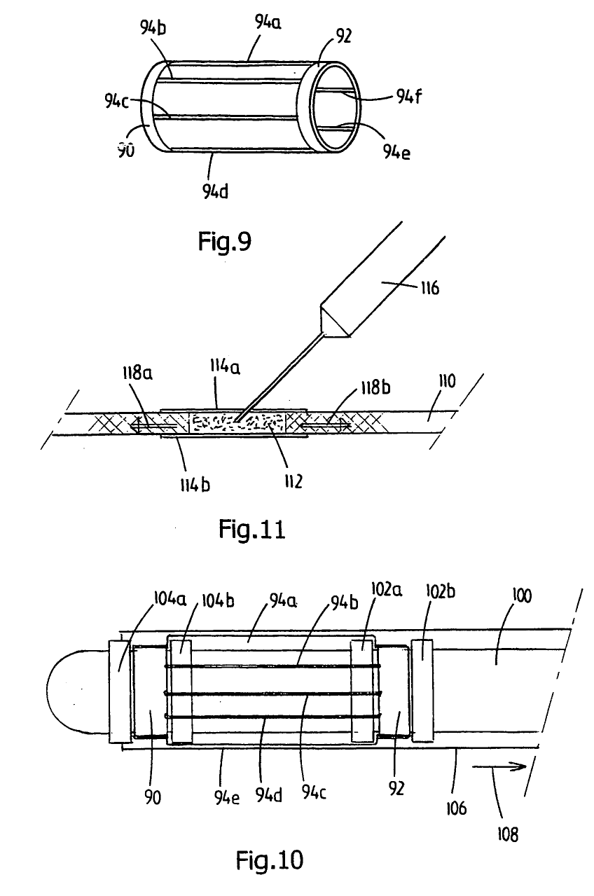

FIG. 9 a perspective view of a fourth embodiment of the invention;

FIG. 10 the distal end of a catheter, suited for positioning the assembly as shown in FIG. 9;

FIG. 11 a part of the wall of a fifth embodiment.

FIG. 1 shows a longitudinal cross-section of a blood vessel 2 at the place of a stenosis (narrowing of the vessel) 4 and shows the situation as it is after a dotter treatment (widening of the restriction). As described in extenso in the publication WO 91/12779, mentioned hereinabove, at the place of aneurism 4 tissue rests 6 remain, consisting of threads of tissue, ripped apart during the dotter treatment, which protrude into the lumen 8 of the blood vessel 2. As FIG. 2 shows it is common practice to position, after the dotter treatment, at the place of this stenosis 4 in het lumen 8 of the blood vessel a stent 10, which is fixed in the blood vessel 2 because the stent 10, is after its positioning, expanded radially outwardly and thus clamped against the vessel wall 2.

Thus these results within the stent 10 an uninterrupted cylindrical passage 12 through which the blood can flow unhindered, and the tissue remains 6 are enclosed between the inner wall 2a of the blood vessel and the cylindrical outer wall 10a of the stent 10. The stent thus exerts on the inner wall 2a of the blood vessel 2 radial forces F1 which keep the stent in place but, of course, also radial forces F2 which are exerted on the tissue remains 6. Also when when only a therapeutically active stent is placed and there has been no dotter treatment this situation occurs.

The invention is based on the insight that these forces F2 are the main cause for the fact that in many cases the result of a dotter treatment, followed by the placing of a stent, are disappointing, so that then the patient has to undergo a new treatment at the same place after a, often disappointingly short, period.

The implant assembly proposed by the invention obviates this problem. FIG. 3 shows a first embodiment of such an assembly and this consists of a cylindrical anchoring element 20, with, fixed thereto, a cylindrical sleeve from a material with a non-rigid structure, such as a supple, flexible, and yielding material, but, also for instance a very “flabby” material which is prepared in such a way—known in itself—that it can deliver in a controlled way a therapeutically active substance such as for instance aspirin or heparin—possibly only from the side opposite the vessel wall. The anchoring element can for instance be the well-known ring, which is radially epandable by means of an expanding balloon, or is made from controlled curing plastics, said curing being initiated, as known in itself, by radiation with ultraviolet radiation. This radiation with ultraviolet radiation can take place when the assembly is positioned by means of a positioning catheter assembly in the blood vessel but it is also possible—when the curing rate as well as the time necessary to position the ring are known—to initiate th curing by irridation still outside the body.

The embodiment according to FIG. 3 can be used when the assembly is placed in a body vessel in which there is always a certain pressure and in which the current of body fluid is always directed in one direction. More universally usable however is an embodiment according to FIG. 4 which is provided with two anchoring elements 26 and 28 respectively, with therebetween the cylindrical section 30 which has the properties as discussed above with regard to the section 22.

FIG. 5 shows an embodiment according to FIG. 4 in which the material section 30′ between the anchoring elements 26 and 28 is provided with cut-outs 32 via which the blood in the blood vessel can reach the tissue under this section.

FIG. 6 shows an embodiment with two anchoring elements 40, 42, similar to the anchoring elements 26, 28 as described above and carrying inbetween strip-shaped sections 44 each having the structure as described above with regard to the section 30 and 30′ respectively.

FIG. 7 shows the effect obtained with the assembly according to the invention. This figure shows a blood vessel 50 with a stenosis 52 and at the position thereof tissue remains 54 which protrude into the lumen 56 of the blood vessel. At the position of this stenosis an assembly according to the invention is placed, consisting of the anchoring elements 58 and 60 respectively with therein between the section 62 which has, as known in itself, such a structure that it delivers in a controlled way a therapeutically active substance such as aspirin or heparin. This section can also be charged to be radioactive or can carry a radioactive source. By a correct choice of the distance between the anchoring elements 58 and 60 it is ensured that, as shown, the cylindrical section 62 lies closely against the tissue remains 54 without, however, exerting an important radial pressure thereon so that the detrimental side effects, mentioned above, will not take place.

To position an assembly according to the invention at its correct position one can use the common positioning catheter of which the end is modified in the way as shown in FIG. 8. This figure shows the end 70 of a positioning catheter provided with an auxiliary carrier 72; this auxiliary carrier 72 carries on its outer surface at exactly pre-determined distances a short first expanding balloon 74 and a second short expanding balloon 76; the balloon 74 carries the expandable ring 78 and the balloon 76 carries the expandable ring 80. Therebetween lies the section 62. As described herein before the correct choice of the distance between the anchoring rings 78 and 80 results in that the pressure exerted by the material section 62 on the tissue 54 thereunder is determined unabiguously and can never be so great that the undesired side effects can occur.

For all the embodiments described above goes that the section which carries the therapeutically active substance can be biologically degradable so that after a certain, known, time period it is completely taken up by the blood or body fluid so that only one or at most two anchoring elements remain.

FIG. 9 shows an embodiment which is more or less derived from the embodiment as shown in FIG. 6; this embodiment has the ring-shaped anchoring elements 90, 92 with therebetween wires 94a . . . 94f which are not intended to provide a support for therapeutically active material but which are themselves made up from therapeutically active material which is also biologically degradable. With modern fabrication techniques it is, indeed, possible to provide therapeutically active material in the shape of thin wires, resulting in a very simple implant assembly indeed.

Of course such a structure is not very rigid and must consequently be handled with great care. To this end the invention proposes to use a special catheter assembly which is shown in detail in FIG. 10. The, elongate, catheter body 100 is provided at its distal end with two pairs of protruding ridges, one pair consisting of the ridges 102a, 102b and the other pair of the ridges 104a, 104b. One ring-shaped anchoring element 90 is accommodated between the ridges 104a, 104b and the other ring-shaped anchoring element 92 is accommodated between the ring-shaped riges 102a, 102b. They can consist of memory metal in the way as known in itself but they can also, each of them, cooperate with a short expanding balloon, not shown in FIG. 10 but being of the kind as discussed on the hand of FIG. 8. An elongate protecting sleeve 106 which can be withdrawn in the direction of the arrow 108 encloses and protects the whole assembly.

After positioning the assembly at the desired spot in the body vessel the sleeve 106 is withdrawn in the direction of the arrow 108; and thereafter the anchoring elements 90 en 92 are expanded, fixing the assembly in place. As time goes by the wire shaped sections 94a . . . 94f deliver their therapeutically active material to their surroudings and are therafter broken up, so that only the two narrow rings 90 and 92 remain in the body vessel.

Finally FIG. 11 elucidates a favourable aspect of the invention. According to this aspect the therapeutically active material is not present in the assembly from the beginning, but is only introduced therein shortly before placing the assembly at its destination in the body vessel. The wall of the section of the implant assembly which carries the therapeutically active material is denoted with reference numeral 110; in this wall a pocket 112 is formed, delimited by the material of the wall and by two thin foils 114a, 114b. Shortly before use of the assembly the therapeutcally active material is introduced into the pocket 112 by means of the syringe 116 and therafter this material spreads in all directions (schematically indicated by the arrows 118a, 118b) through the section 110 so that this therapeutically active material can be delivered to its surroundings when the assembly has been put into place. So the amount of therapeutically active material is decided upon at the last moment, taking all considerations into account.

Within the framework of the invention many modifications are possible with regard to the structure of the therapeutically active section which can be without any problems be designed such that the active substances are delivered well-dosed and during a long time period. Furthermore all measures and techniques, known in practice for the treatment of body vessel disorders, can be put into effect.

1. Implant assembly to be fixed in place in a body vessel, comprising a section which is directed in the direction of the vessel axis and is provided with means to act upon the surrounding tissue, characterized in that this section can be anchored with at least one end by means of an anchoring element to the vessel wall and has a non-rigid structure, in such a way that it can adapt itself to the vessel wall configuration without exerting apprecible radial pressure on the surrounding tissue.

2. Implant assembly according to claim 1, characterized in that the section has a cylinder shape.

3. Implant assembly according to claim 1, characterized in that het section is strip-shaped and is connected together, with a number of similar sections, to the circumference of the anchoring element.

4. Implant assembly according to claims 1-3, characterized in that the anchoring element is a radially expandable ring.

5. Implant assembly according to claims 1-4, characterized in that the section carries a therapeutically active substance and is structured for a dosed delivery thereof.

6. Implant assembly according to claim 5, characterized in that the substance is present at the side which is opposite the vessel wall.

7. Implant assembly according to claims 1-6, characterized in that the section carries a source of ionising radiation.

8. Implant assembly according to claims 1-7, characterized by cut-outs provided in the surface of the sections.

9. Implant assembly according to claims 1-8, characterized in that the section consists of biologically degradable material.

10. Implant assembly according to claims 1-9, characterized in that the active material carrying section has the shape of a wire-like element, connected with at least, one end to an anchoring element.

11. Implant assembly according to claims 1-9, characterized in that the or each section is provided with one or more pockets, accessible from the outside, for instance by means of a syringe, to be filled with a therapeutically active substance prior to th positioning thereof in the body vessel.

12. Implant assembly according to claims 1-9, characterized in that the section is made from flexibly yielding material.

13. Implant assembly according to claims 1-10, characterized by two anchoring elements, with the section or sections provided therein between.

14. Catheter to be used in combination with an implant assembly according to claim 10, characterized in that the distal end thereof is provided with two pairs of surrounding ridges, protruding from the catheter body, the respective regions of each pair accommodating between them a ring-anchoring element which is either self-expanding or cooperates with an expansion element the whole being enclosed by a removable protecting sleeve.

15. A method for positioning an implant assembly according to claim 14 in a body vessel, in which the pre-tension in the section, and therewith the radial pressure exerted thereby on its surrounding, is adjusted to a predetermined value by the choice of the mutual distance of the anchoring elements.
